(19) United States
(12) Patent Application Publication
(10) Pub. No.: US 2025/0345266 A1
(43) Pub. Date: Nov. 13, 2025

(54) READY-TO-INFUSE OXYTOCIN COMPOSITIONS

(71) Applicant: AMNEAL PHARMACEUTICALS LLC, Bridgewater, NJ (US)

(72) Inventors: Srinivas Ramachandra Rao Kone, Monroe Township, NJ (US); Tarun Goswami, Plainsboro, NJ (US); Harsha Vardhan Bandarupalli, Middletown, NJ (US); Hardik Pravinbhai Patel, Hillsborough, NJ (US); SUHASKUMAR Patel, Edison, NJ (US)

(73) Assignee: AMNEAL PHARMACEUTICALS LLC, Bridgewater, NJ (US)

(21) Appl. No.: 19/202,110

(22) Filed: May 8, 2025

Related U.S. Application Data
(60) Provisional application No. 63/644,101, filed on May 8, 2024.

(51) Int. Cl. A61K 9/00; A61K 9/08; A61K 38/095; A61K 47/02; A61K 47/18; A61K 47/22

(52) U.S. Cl. CPC A61K 9/0019; A61K 9/08; A61K 38/095; A61K 47/02; A61K 47/183; A61K 47/22

(57) ABSTRACT

The present disclosure provides aqueous, ready-to-infuse oxytocin injection compositions comprising from about 0.01 IU/mL to about 0.1 IU/mL oxytocin, chelating agent, buffer, and aspartic acid, wherein the composition is free of any preservative, and comprises an oxytocin: aspartic acid wt/vol ratio of from about 1:50 to about 1:2000.

### CROSS REFERENCE

This application claims the benefit of priority to U.S. Provisional Patent Application No. 63/644,101, filed on May 8, 2024, the disclosure of which is hereby incorporated by reference in its entirety.

### TECHNICAL FIELD

The present invention relates to aqueous, ready-to-infuse oxytocin injection compositions and methods for preparing the same. The ready-to-infuse oxytocin injection compositions of the disclosure minimize human error and fatalities associated with errors in dilution.

### BACKGROUND

Oxytocin is a cyclic nonapeptide with a half-life of about 3 minutes. It is secreted by the posterior pituitary gland that causes uterine contraction by binding to oxytonergic receptors. It undergoes rapid degradation to inactive products when exposed to elevated temperature and therefore requires storage below 25° C. Clinically, oxytocin is often used to induce labor and support labor in case of non-progression of parturition and to treat (post-partum) hemorrhage. Currently, oxytocin is considered to be the principal agent to treat post-partum hemorrhage. It is degraded in the gastrointestinal (GI) tract and is therefore administered as an aqueous formulation by injection or as nasal spray. The empirical formula of oxytocin is C43H66N1201252; and molecular weight is about 1007.2. The molecule includes the following nonapeptide sequence with a disulfide bridge between the two cysteine residues at positions 1 and 6.
Cys-Tyr-Ile-Gln-Asn-Cys-Pro-Leu-Gly

Complications related to pregnancy and childbirth claim nearly 800 women's lives each day. This tragedy is compounded by the fact that the vast majority of these deaths are preventable. However, ninety-nine percent of maternal deaths occur in the developing world where basic medical services and supplies are typically scarce. Postpartum hemorrhage (PPH; excessive bleeding after childbirth), accounts for nearly 25% of maternal deaths globally, and is an especially acute concern in resource-scarce settings. The pharmacological treatment recommended for PPH by the World Health Organization, e.g., treatment with oxytocin, is incompatible with regions where reliable refrigeration is unavailable. Typically formulated as an aqueous solution for injection, oxytocin rapidly loses efficacy at room temperature via chemical degradation. While degradation may not necessarily be a safety issue, specifically in the case of oxytocin (Hodgins and Lukulay, Int J Gynaecol Obstet. 2017, 136 (3): 253-254), it can still lead to a reduced active pharmaceutical ingredient level and hence compromised performance. Further the review literature revisiting the Stability and Storage Specifications of Oxytocin Injection (USAID, July 2018), provides insight on assay drops in various marketed oxytocin formulations worldwide. The pharmacopoeia limits of assay in the range of 90-110% as accepted by various standards is critical to meet for such formulation. Furthermore, dilute solutions of oxytocin generally have a significantly shorter shelf-life than concentrated oxytocin solutions.

Oxytocin is currently available in the United States as a single use 1 ml glass vial (PITOCIN®). The formulation is typically present at a concentration of 10 United States Pharmacopoeia (USP) Units/ml (10 IU/mL). The available formulation contains chlorobutanol at 0.5% (5 mg/ml) as a preservative and has a pH of 3.0 to 5.0. The method of administration of oxytocin is through intravenous (IV) infusion. To prepare the usual solution for infusion, 1-mL Oxytocin Injection, 10 IU/mL is combined aseptically with 1,000 mL of non-hydrating diluent (i.e., 0.9% Sodium chloride-physiologic electrolyte solution) providing an oxytocin concentration of about 10 mU/ml (e.g., 1 USP Unit/100 mL). The combined solution is then rotated in the infusion bottle to ensure thorough mixing. Then, this dilute oxytocin solution is delivered to a patient through use of a constant infusion pump or another similar device to accurately control the rate of infusion. The current formulation requires multiple steps of mixing, and adjustment of administration rate of oxytocin for correlation to an administration rate of about 0.5-1 mU/min.

Intranasal oxytocin (SYNTOCINON®) had been approved by the U.S. Food and Drug Administration (US FDA) for stimulating milk letdown to facilitate breast-feeding from 1960 until 1997. While the nasal spray of SYNTOCINON® was withdrawn from the U.S. market at the request of the manufacturer, intranasal oxytocin is still marketed outside of the United States in countries such as Switzerland, Portugal, or Brazil.

U.S2019/388,498 discloses aqueous ready-to-use formulations of oxytocin. The formulations are free of chlorobutanol and contain high concentrations of oxytocin. The formulations are stabilized by various stabilizing agents, for example, sodium edetate, and amino acids. The publication, however, fails to provide any stability data, much less long-term storage stability under refrigerated and controlled room temperature storage conditions. WO2022167978 discloses ready-to-infuse stable parenteral dosage forms of oxytocin. The dosage forms are solutions comprising disaccharides, sodium acetate and other excipients. U.S. Pat. No. 11,389,473 discloses formulations comprising oxytocin and magnesium salt. The patent further discloses methods for the treatment of pain (such as migraine headache) comprising co-administration of an oxytocin peptide and a magnesium salt.

There is an unmet need for stable, ready-to-infuse oxytocin injection compositions that minimize human errors and fatalities associated with the errors in dilution, microbial infections during dilution, and errors during adjustment of the administration rate to provide an initial administration rate of about 0.5-1 mU/min (0.0005 IU/min-0.001 IU/min) that can be gradually increased in increments of 1-2 mU/min until the desired contraction pattern has been established.

### SUMMARY

In one embodiment, the invention relates to an aqueous, ready-to-infuse oxytocin injection composition comprising from about 0.01 IU/mL to 0.1 IU/mL of oxytocin, a chelating agent, and an amino acid; wherein the composition comprises oxytocin: chelating agent wt/vol ratio of from about 1:10 to about 1:750. The composition contains at least about 90% wt/vol of oxytocin assay after storage for at least about 3 months under refrigerated and controlled room temperature conditions.

In certain embodiments, the oxytocin: amino acid wt/vol ratio is from about 1:50 to about 1:2000.

In certain embodiments, the refrigerated conditions comprise a temperature of about 2-8° C. and the controlled room temperature conditions comprise a temperature of 25° C.±5° C. and 40%±5% relative humidity.

In certain embodiments, the chelating agent is selected from the group consisting of 1,4,7,10-tetraazacyclododecane-1,4,7,10-tetraacetic acid (DOTA), diethylene triamine-N,N,N′,N″,N″-pentaacetate/pentetic acid (DTPA), ethylenediamine tetraacetic acid (EDTA), calcium disodium edetate or their salts, and mixtures thereof. In certain embodiments, the chelating agent is 1,4,7,10-tetraazacyclododecane-1,4,7,10-tetraacetic acid (DOTA).

In certain embodiments, the amino acid is selected from the group consisting of arginine, glycine, alanine, proline, methionine, lysine, leucine, cysteine, aspartic acid, isoleucine, and mixtures thereof. In certain embodiments, the amino acid is L-aspartic acid.

In certain embodiments, the composition further comprises sodium chloride.

In certain embodiments, the composition further comprises a buffer selected from the group consisting of phosphate buffer, citrate buffer, sodium carbonate, sodium bicarbonate, tartrate, benzoate, ascorbic acid, succinic acid, lactic acid, glutaric acid, malic acid, boric acid, orthophosphoric acid and carbonic acid, alkali or alkaline earth salt of one of these acids, and mixtures thereof.

In certain embodiments, the composition does not include a preservative.

In certain embodiments, the composition exhibits a pH of between 3.5 and 5.5.

In another embodiment, the invention relates to an aqueous, ready-to-infuse oxytocin injection comprising from about 0.01 IU to 0.1 IU/mL of oxytocin, a chelating agent, and aspartic acid, wherein the composition comprises oxytocin: aspartic acid wt/vol ratio of from about 1:50 to about 1:2000, and wherein the composition does not include a preservative. The composition contains at least about 90% wt/vol of oxytocin assay after storage for at least about 3 months under refrigerated and controlled room temperature conditions.

In certain embodiments, the oxytocin: chelating agent wt/vol ratio is from about 1:10 to about 1:750.

In certain embodiments, the composition contains at least about 95% wt/vol of oxytocin assay after storage for at least about 3 months under refrigerated and controlled room temperature conditions. In certain embodiments, the refrigerated conditions comprise a temperature of about 2-8° C. and the controlled room temperature conditions comprise a temperature of 25° C.±2° C. and 40%±5% relative humidity.

In certain embodiments, the composition comprises from about 0.000016 mg/ml to about 0.0001 mg/ml of oxytocin.

In certain embodiments, the composition further comprises a buffer selected from the group consisting of phosphate buffer, citrate buffer, sodium carbonate, sodium bicarbonate, tartrate, benzoate, ascorbic acid, succinic acid, lactic acid, glutaric acid, malic acid, boric acid, orthophosphoric acid and carbonic acid, alkali or alkaline earth salt of one of these acids, and mixtures thereof.

In certain embodiments, the chelating agent is 1,4,7,10-tetraazacyclododecane-1,4,7,10-tetraacetic acid.

In certain embodiments, the composition exhibits a pH of between 3.5 and 5.5.

In still another embodiment, the invention relates to an aqueous, ready-to-infuse oxytocin injection composition comprising from about 0.01 IU to 0.1 IU/mL of oxytocin, a chelating agent, and an amino acid, wherein the composition comprises oxytocin: aspartic acid wt/vol ratio of from about 1:50 to about 1:2000, and the oxytocin: chelating agent wt/vol ratio of from about 1:10 to about 1:750. The composition contains at least about 90% wt/vol of oxytocin assay after storage for at least about 3 months under refrigerated and controlled room temperature conditions.

In certain embodiments, the composition contains at least about 95% wt/vol of oxytocin assay after storage for at least about 3 months under refrigerated and controlled room temperature conditions.

In another embodiment, the invention relates to a method for initiation or improvement of uterine contractions to achieve vaginal delivery in a subject with a medical indication for the initiation or improvement of labor, the method comprising administering to the subject an aqueous, ready-to-infuse oxytocin injection composition comprising from about 0.01 IU to 0.1 IU/mL of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

In certain embodiments, the refrigerated conditions comprise a temperature of about 2-8° C. and the controlled room temperature conditions comprise a temperature of 25° C.±2° C. and 40%±5% relative humidity.

In another embodiment, the invention relates to a method for induction of labor in a subject with a medical indication for initiation of labor, the method comprising administering to the subject an aqueous, ready-to-infuse oxytocin injection composition comprising from about 0.01 IU to 0.1 IU/ml of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

In certain embodiments, the refrigerated conditions comprise a temperature of about 2-8° C. and the controlled room temperature conditions comprise a temperature of 25° C.±2° C. and 40%±5% relative humidity.

In certain embodiments, the medical indication comprises Rh problems, maternal diabetes, preeclampsia at or near term, when delivery is in the best interests of mother and fetus or when membranes are prematurely ruptured, and delivery is indicated.

In another embodiment, the invention relates to a method for stimulation or reinforcement of labor in a subject with uterine inertia, the method comprising administering to the subject an aqueous, ready-to-infuse oxytocin injection composition comprising from about 0.01 IU to 0.1 IU/ml of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

In certain embodiments, the refrigerated conditions comprise a temperature of about 2-8° C. and the controlled room temperature conditions comprise a temperature of 25° C.±2° C. and 40%±5% relative humidity.

In still another embodiment, the invention relates to a method for management of incomplete or inevitable abortion in a subject in need thereof, the method comprising administering to the subject an aqueous, ready-to-infuse oxytocin injection composition comprising from about 0.01 IU to 0.1 IU/ml of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions. The ready-to-infuse oxytocin injection composition is administered as an adjunct to a primary therapy in the management of incomplete or inevitable abortion.

In certain embodiments, the primary therapy comprises curettage in first trimester of the subject.

In certain embodiments, the primary therapy comprises emptying of the uterus in second trimester.

In certain embodiments, the refrigerated conditions comprise a temperature of about 2-8° C. and the controlled room temperature conditions comprise a temperature of 25° C.±2° C. and 40%±5% relative humidity.

In another embodiment, the invention relates to a method for controlling postpartum bleeding or hemorrhage in a subject in need thereof, the method comprising administering to the subject an aqueous, ready-to-infuse oxytocin injection composition comprising from about 0.01 IU to 0.1 IU/ml of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

In certain embodiments, the refrigerated conditions comprise a temperature of about 2-8° C. and the controlled room temperature conditions comprise a temperature of 25° C.±2° C. and 40%±5% relative humidity.

In another embodiment, the disclosure provides a method for producing uterine contractions during third stage of labor in a subject, the method comprising administering to the subject an aqueous, ready-to-infuse oxytocin injection composition comprising from about 0.01 IU to 0.1 IU/ml of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

In certain embodiments, the refrigerated conditions comprise a temperature of about 2-8° C. and the controlled room temperature conditions comprise a temperature of 25° C.±2° C. and 40%±5% relative humidity.

In another embodiment, the disclosure provides a method for induction of labor in a subject with a medical indication for initiation of labor, the method comprising administering to the subject an aqueous, ready-to-infuse oxytocin injection composition at an initial administration rate of 0.5-1 mU/min for up to 30 minutes, gradually increasing the administration rate to 1-2 mU/min from 30-60 minutes to obtain a desired contraction pattern and progression of labor to 5-6 cm dilation; wherein the composition comprises from about 0.01 IU to 0.1 IU/mL of oxytocin, a chelating agent, and aspartic acid; wherein the composition comprises oxytocin: aspartic acid wt/vol ratio of from about 1:50 to about 1:2000; and wherein the composition does not include a preservative. The composition contains at least about 90% wt/vol of oxytocin assay after storage for at least about 3 months under refrigerated and controlled room temperature conditions.

### BRIEF DESCRIPTION OF DRAWINGS

FIG. 1 provides a flow chart of the manufacturing process used for making ready-to infuse oxytocin compositions of the disclosure.

### DETAILED DESCRIPTION

The following detailed description is exemplary and explanatory and is intended to provide further explanation of the present disclosure described herein. Other advantages, and novel features will be readily apparent to one of ordinary skill in the art from the following detailed description of the present disclosure.

The present disclosure provides aqueous, ready-to-infuse oxytocin injection compositions comprising oxytocin and at least one stabilizer. In certain embodiments, the stabilizer comprises a buffer, a chelating agent, and/or an amino acid. In certain embodiments, the aqueous, ready-to-infuse oxytocin injection compositions comprise oxytocin, an amino acid, and a chelating agent. In certain embodiments, the aqueous, ready-to-infuse oxytocin injection compositions of the disclosure include oxytocin: amino acid ratio of from about 1:50 to about 1:2000, and oxytocin: chelating agent ratio of from about 1:10 to about 1:750. In certain embodiments, the oxytocin: amino acid ratio and oxytocin: amino acid ratios are wt/vol ratios. In certain embodiments, the oxytocin: amino acid ratio and oxytocin: amino acid ratios are wt/wt ratios.

In certain embodiments, the aqueous, ready-to-infuse oxytocin injection compositions exhibit excellent storage stability for at least about 3 months under long term and refrigerated storage conditions, with a level of any single impurity of less than 3% by weight and a level of total impurities of less than 10% by weight.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification.

Unless otherwise defined, all technical and scientific terms used herein have the same meaning as commonly understood by one of the ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

As used herein, the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” or “comprising” are interchangeable, and one of skill in the art is cognizant that these terms are open-ended terms.

The words “comprise”, “comprises”, and “comprising” are to be interpreted inclusively rather than exclusively. The words “consist,” “consisting,” and its variants, are to be interpreted exclusively, rather than inclusively.

As used herein, the term “and/or” refers to and encompasses any and all possible combinations of one or more of associated listed items.

As used herein, the terms “about” or “approximately,” mean a variability of 10% or less from the reference given, unless otherwise specified.

As used herein, the term “free” means the composition, formulation or material does not contain the component modified by the term “free.”

As used herein, the term “substantially free” refers to excluding any functional (e.g., noncontaminating) amount, which refers to any amount that contributes/influences therapeutic efficacy/effect of the composition.

As used herein the term “non-aqueous” should be accorded its normal meaning which may mean the composition, formulation or material does not contain any added or additional water other than the amount of water that commonly associated with the non-water components present in the composition, formulation or material. For example, a non-aqueous composition comprising solvents such as glycerin or propylene glycol will contain trace amounts of water because the United States Pharmacopeia allows glycerin to contain up to 5.0% water and propylene glycol to contain up to 0.2% water.

As used herein, the term “aqueous” should be accorded its normal meaning which may mean the composition, formulation or material that contains water as a solvent or medium.

As used herein, the term “oxytocin” includes the original nonapeptide having the amino acid sequence as described above.

The term ‘ready-to-infuse,’ as used herein, refers to aqueous solution of oxytocin that is sterile and suitable for direct intravenous infusion without manipulation, that is, no intermediate steps of dilution, reconstitution, dispensing, sterilization, transfer, handling or compounding are required before administration or infusion of the solution to the patient. The aqueous solution can be directly administered parenterally from the container/bag. Also, a ready-to-infuse parenteral dosage form can be said to be a premixed dosage form which can be administered directly without any dilution or mixing requirement.

The terms “pH adjusting agent,” and “buffer,” as used interchangeably herein, refer to acids and bases that modify and/or stabilize the acidity or alkalinity (pH) of the composition formulation. Commonly used pH adjusting agents/buffering agents include phosphate buffer, citrate buffer, sodium carbonate, sodium bicarbonate, tartrate, benzoate, ascorbic acid, succinic acid, lactic acid, glutaric acid, malic acid, boric acid, orthophosphoric acid and carbonic acid, alkali or alkaline earth salt of one of these acids, and mixtures thereof.

The term “stable” as used herein refers to a ready-to-infuse aqueous solution composition comprising oxytocin, wherein the solution does not change physically and about 90 to about 110 percent of the assay (oxytocin amount based on the dose amount/strength of oxytocin in the composition) remains unchanged after storage under refrigerated conditions (2-8° C.) or controlled room temperature conditions (25° C.±5° C. and 40%±5% relative humidity) for at least about 3 months, preferably about 6 months to about 12 months.

As used interchangeably herein, the terms “accelerated condition” and “accelerated storage condition” refer to a temperature of 40° C.±2° C. and 25±5% relative humidity (RH).

When stored under these conditions, the aqueous, ready-to-infuse oxytocin injection composition remains chemically stable, wherein various parameters such a drug content (% assay of oxytocin) and content of related substances, i.e. known impurities, unknown impurities and total impurity, remains within specified limits. Suitably, the assay of oxytocin remains within 90%-110% by weight of the oxytocin dose amount (oxytocin assay prior to storage), the content of total impurities remains within 10% by weight and the content of any single known impurity remains within 3% by weight (relative to oxytocin assay prior to storage). The assay may be determined using HPLC technique.

As used herein, the term “long-term storage” is understood to mean that the ready-to-infuse aqueous solution composition can be stored for at least about three months, at least about six months, and preferably for one year or more, most preferably a minimum stable shelf life of at least 18 months. Generally speaking, the terms “long term storage” further include stable storage durations that are at least comparable to or better that the stable shelf life typically required for currently available commercial formulations of oxytocin, without losses in stability that would render the formulation unsuitable for its intended pharmaceutical application. Long-term storage is also understood to mean that the pharmaceutical composition is stored either as a liquid at 2-8° C., or controlled room temperature condition (e.g., 25° C.).

### Ready-to-Infuse Oxytocin Compositions

The present disclosure provides aqueous, ready-to-infuse oxytocin injection compositions comprising oxytocin (oxytocin compositions). In particular, the disclosure provides Oxytocin

Injection, USP, Synthetic, 0.010 IU/mL, 0.015 IU/mL, 0.020 IU/mL, 0.025 IU/mL, 0.030 IU/mL, 0.035 IU/mL, 0.04 IU/mL, 0.045 IU/mL, 0.05 IU/mL, 0.055 IU/mL, 0.06 IU/mL, 0.065 IU/mL, 0.07 IU/mL, 0.075 IU/mL, 0.08 IU/mL, 0.085 IU/mL, 090 IU/mL, 0.095 IU/mL, or 0.10 IU/mL as a premixed, ready-to-use aqueous solution for intravenous infusion. No further dilution of these preparations is necessary.

In certain embodiments, the disclosure provides aqueous, ready-to-infuse oxytocin injection compositions comprising Oxytocin Injection, USP, Synthetic, 10 mU/mL, 15 mU/mL, 20 mU/mL, 25 mU/mL, 30 mU/mL, 35 mU/mL, 40 mU/mL, 45 mU/mL, 50 mU/mL, 55 mU/mL, 60 mU/mL, 65 mU/mL, 70 mU/mL, 75 mU/mL, 80 mU/mL, 85 mU/mL, 90 mU/mL, 95 mU/mL, or 100 mU/mL as a premixed, ready-to-use aqueous solution for intravenousinfusion. No further dilution of these preparations is necessary.

In certain embodiments, the disclosure provides aqueous, ready-to-infuse oxytocin injection compositions comprising 0.01 IU/mL to 0.10 IU/mL oxytocin, amino acid, pH adjusting agent, chelating agent, and tonicity agent. In certain embodiments, the aqueous, ready-to-infuse oxytocin injection compositions are preservative free compositions. In certain embodiments, the presence of preservative is optional.

Peptides are inherently unstable substances that require specific excipients for imparting stability to the formulation. Stabilizers play a major role in maintaining the stability of the peptides, particularly those in solution form. According to embodiments of the present invention, stabilizers include excipients selected from an acid pH adjusting agent, an amino acid, and/or a chelating agent.

In certain embodiments, the aqueous, ready-to-infuse oxytocin injection composition comprises amino acid as a stabilizer, wherein the amino acid is selected from the group consisting of arginine, glycine, alanine, proline, methionine, lysine, leucine, cysteine, L-aspartic acid, isoleucine, and mixtures thereof.

In certain embodiments, the amino acid is present in an amount of about 0.0001% wt/vol, about 0.0002% wt/vol, about 0.0003% wt/vol, about 0.0004% wt/vol, about 0.0005% wt/vol, about 0.0005% wt/vol, about 0.0007% wt/vol, about 0.0008% wt/vol, about 0.0009% wt/vol, about 0.001% wt/vol, about 0.002% wt/vol, about 0.003% wt/vol, about 0.004% wt/vol, about 0.005% wt/vol, about 0.006% wt/vol, about 0.007% wt/vol, about 0.008% wt/vol, about 0.009% wt/vol, about 0.01% wt/vol, about 0.02% wt/vol, about 0.03% wt/vol, about 0.04% wt/vol, about 0.05% wt/vol, about 0.06% wt/vol, about 0.07% wt/vol, about 0.08% wt/vol, about 0.09% wt/vol, about 0.1% wt/vol, or any intermediate values therein of the ready-to-use aqueous solution for intravenous infusion.

In certain embodiments, the amino acid is present in an amount of about 0.001 mg/ml, about 0.002 mg/ml, about 0.003 mg/ml, about 0.004 mg/ml, about 0.005 mg/ml, about 0.005 mg/ml, about 0.007 mg/ml, about 0.008 mg/ml, about 0.009 mg/ml, about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, or any intermediate values therein of the ready-to-use aqueous solution for intravenous infusion. In certain embodiments, the amino acid is aspartic acid. In certain embodiments, the amino acid is L-aspartic acid present in an amount of from about 0.001 mg/ml to about 0.1 mg/ml, preferably, from about 0.001 to about 0.05 mg/ml of the ready-to-use aqueous solution for intravenous infusion.

In certain embodiments, the disclosure provides aqueous, ready-to-infuse oxytocin injection compositions comprising oxytocin and amino acid in a wt/vol or wt/wt ratio of from about 1:50 to about 1:2000, about 1:100, about 1:150, about 1:200, about 1:250, about 1:300, about 1;350, about 1:400, about 1:450, about 1:500, about 1:550, about 1:600, about 1;650, about 1:700, about 1:750,about 1:800, about 1:850, about 1:900, about 1:950, about 1:1000, about 1:1050, about 1:1100,about 1:1150, about 1:1200, about 1:1250, about 1:1300, about 1:1350, about 1:1400, about 1:1450, about 1:1500, about 1:1550, about 1:1600, 1:1650, about 1:1700, about 1:1750, about 1:1800, about 1:1850, about 1:1900, about 1:1950, about 1:2000, or any intermediate ratios therein. In certain embodiments, the aqueous, ready-to-infuse injection compositions of the disclosure comprise oxytocin and amino acid in a wt/vol or wt/wt ratio of from about 1:50 to about 1:1500.

In certain embodiments the amino acid is aspartic acid. In certain embodiments the amino acid is L-aspartic acid.

In certain embodiments, the aqueous, ready-to-infuse oxytocin injection composition comprises an acid pH adjusting agent as a stabilizer, wherein the acid is selected from the group consisting of citric acid, ascorbic acid, succinic acid, lactic acid, glutaric acid, malic acid, boric acid, orthophosphoric acid and carbonic acid, and mixtures thereof. In certain embodiments, the acid pH adjusting agent is boric acid.

In certain embodiments, the aqueous, ready-to-infuse oxytocin injection compositions exhibit a pH of from about 3.5 to about 5.5. In certain embodiments, the aqueous, ready-to-infuse oxytocin injection compositions exhibit a pH of about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5 or any intermediate values therein. In certain embodiments, the acid pH adjusting agent is present in an amount of about 0.0001% wt/vol, about 0.0002% wt/vol, about 0.0003% wt/vol, about 0.0004% wt/vol, about 0.0005% wt/vol, about 0.0005% wt/vol, about 0.0007% wt/vol, about 0.0008% wt/vol, about 0.0009% wt/vol, about 0.001% wt/vol, about 0.002% wt/vol, about 0.003% wt/vol, about 0.004% wt/vol, about 0.005% wt/vol, about 0.006% wt/vol, about 0.007% wt/vol, about 0.008% wt/vol, about 0.009% wt/vol, about 0.01% wt/vol, about 0.02% wt/vol, about 0.03% wt/vol, about 0.04% wt/vol, about 0.05% wt/vol, about 0.06% wt/vol, about 0.07% wt/vol, about 0.08% wt/vol, about 0.09% wt/vol, about 0.1% wt/vol, or any intermediate values therein, of the ready-to-use aqueous solution for intravenous infusion. In certain embodiments, the boric acid is present in an amount of from 0.001% wt/vol to about 0.1% wt/vol of the ready-to-use aqueous solution for intravenous infusion.

In certain embodiments, the pH adjusting agent is present in an amount to provide ready-to-infuse oxytocin injection compositions with a pH of from about 3.0 to about 5.5.

In certain embodiments, the stabilizer is a chelating agent selected from the group consisting of DOTA (1,4,7,10-tetraazacyclododecane-1,4,7,10-tetraacetic acid), DTPA (diethylene triamine-N,N,N′,N″,N″-pentaacetate)/pentetic acid, EDTA (ethylenediamine tetraacetic acid), calcium disodium edetate or their salts, and mixtures thereof. In certain embodiments, the chelating agent is DOTA. In certain embodiments, the chelating agent is not an amino acid. In certain embodiments, the chelating agent is present in an amount of from about 0.0001% wt/vol to about 0.1% wt/vol of the ready-to-use aqueous solution for intravenous infusion.

In certain embodiments, the chelating agent is present in an amount of about 0.0001% wt/vol, about 0.0002% wt/vol, about 0.0003% wt/vol, about 0.0004% wt/vol, about 0.0005% wt/vol, about 0.0005% wt/vol, about 0.0007% wt/vol, about 0.0008% wt/vol, about 0.0009% wt/vol, about 0.001% wt/vol, about 0.002% wt/vol, about 0.003% wt/vol, about 0.004% wt/vol, about 0.005% wt/vol, about 0.006% wt/vol, about 0.007% wt/vol, about 0.008% wt/vol, about 0.009% wt/vol, about 0.01% wt/vol, about 0.02% wt/vol, about 0.03% wt/vol, about 0.04% wt/vol, about 0.05% wt/vol, about 0.06% wt/vol, about 0.07% wt/vol, about 0.08% wt/vol, about 0.09% wt/vol, about 0.1% wt/vol, or any intermediate values therein, of the ready-to-use aqueous solution for intravenous infusion.

In certain embodiments, the disclosure provides aqueous, ready-to-infuse oxytocin injection compositions comprising oxytocin and chelating agent (e.g., DOTA) in a wt/vol or wt/wt ratio of from about 1:10 to about 1:750, about 1:20, about 1:30, about 1:40, about 1:50, about 1:60, about 1:70, about 1:80, about 1:90, about 1:100, about 1:110, about 1:120, about 1:130, about 1:140,about 1:150, about 1:160, about 1:170, about 1:180, about 1:190, about 1:200, about 1:250, about 1:300, about 1:350, about 1:400, about 1:450, about 1:500, about 1:550, about 1:600, about 1;650,about 1:700, about 1:750, or any intermediate ratios therein.

In certain embodiments, the aqueous, ready-to-infuse injection compositions of the disclosure comprise oxytocin and chelating agent in a wt/vol or wt/wt ratio of from about 1:10 to about 1:600.

In certain embodiments the chelating agent is 1,4,7,10-tetraazacyclododecane-1,4,7,10-tetraacetic acid (DOTA).

In certain embodiments, the stabilizer is a mixture of a chelating agent and an amino acid. In certain embodiments, the amino acid is selected from the group consisting of arginine, glycine, alanine, proline, methionine, lysine, leucine, cysteine, aspartic acid, isoleucine, and mixtures thereof. In certain embodiments, the chelating agent is selected from the group consisting of DOTA (1,4,7,10-tetraazacyclododecane-1,4,7,10-tetraacetic acid), DTPA (diethylene triamine-N,N,N′,N″,N″-pentaacetate)/pentetic acid, EDTA (ethylenediamine tetraacetic acid), calcium disodium edetate or their salts, and mixtures thereof.

In certain embodiments, the stabilizer is a mixture of DOTA and aspartic acid (e.g., L-aspartic acid). In certain embodiments, the mixture of DOTA and L-aspartic acid is present in an amount of about 0.0001% wt/vol, about 0.0002% wt/vol, about 0.0003% wt/vol, about 0.0004% wt/vol, about 0.0005% wt/vol, about 0.0005% wt/vol, about 0.0007% wt/vol, about 0.0008% wt/vol, about 0.0009% wt/vol, about 0.001% wt/vol, about 0.002% wt/vol, about 0.003% wt/vol, about 0.004% wt/vol, about 0.005% wt/vol, about 0.006% wt/vol, about 0.007% wt/vol, about 0.008% wt/vol, about 0.009% wt/vol, about 0.01% wt/vol, about 0.02% wt/vol, about 0.03% wt/vol, about 0.04% wt/vol, about 0.05% wt/vol, about 0.06% wt/vol, about 0.07% wt/vol, about 0.08% wt/vol, about 0.09% wt/vol, about 0.1% wt/vol, or any intermediate values therein, of the ready-to-use aqueous solution for intravenous infusion.

In certain embodiments, the stabilizer is a mixture of a chelating agent and amino acid present in a wt/vol or wt/wt ratio of amino acid: chelating agent of from about 1:1 to about 10:1. In certain embodiments, the amino acid and the chelating agent are present in a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, bout 10:1, or any intermediate ratios therein.

In certain embodiments, the stabilizer is a mixture of an L-aspartic acid and DOTA present in a wt/vol or wt/wt ratio of L-Aspartic acid: DOTA of from about 1:1 to about 10:1. In certain embodiments, the amino acid and the chelating agent are present in a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, bout 10:1, or any intermediate ratios therein.

The aqueous, ready-to-infuse oxytocin injection compositions of the disclosure comprise a tonicity agent elected from the group comprising sodium chloride, dextrose solution, Ringer's lactate solution, and a mixture thereof. In certain embodiments, the tonicity agent is sodium chloride. In certain embodiments, the tonicity agent is present in an amount of from about 0.5% wt/vol to about 2.8% wt/vol of the aqueous, ready-to-infuse aqueous injection composition. In a preferred embodiment, the tonicity agent is sodium chloride. In certain embodiments, the sodium chloride is present in an amount of about 0.9% wt/vol of the solution composition.

In certain embodiments, the aqueous, ready-to-infuse oxytocin injection compositions of the disclosure are preservative free compositions. In certain embodiments, the aqueous, ready-to-infuse oxytocin injection compositions of the disclosure comprise a preservative selected from the group comprising benzyl alcohol, phenol, paraben, chlorobutanol, and mixtures thereof. In certain embodiments, the preservative is chlorobutanol. In certain embodiments, the preservative is present in an amount of from about 0.001 to about 0.5% wt/vol of the parenteral composition. In certain embodiments, the preservative is present in an amount of about 0.001% wt/vol, about 0.002% wt/vol, about 0.003% wt/vol, about 0.004% wt/vol, about 0.005% wt/vol, about 0.006% wt/vol, about 0.007% wt/vol, about 0.008% wt/vol, about 0.009% wt/vol, about 0.01% wt/vol, about 0.015% wt/vol, about 0.02% wt/vol, about 0.025% wt/vol, about 0.03% wt/vol, about 0.035% wt/vol, about 0.04% wt/vol, about 0.045% wt/vol, about 0.05%, or any intermediate values therein.

In certain embodiments, the aqueous, ready-to-infuse oxytocin injection compositions of disclosure are supplied in a fill volume of from about 100 mL to about 1000 mL. In certain embodiments, the ready-to-infuse oxytocin solution compositions are supplied in a fill volume of about 100 mL, about 150 mL, about 200 mL, about 250 mL, about 300 mL, about 350 mL, about 400 mL, about 450 mL, about 500 mL, 550 mL, about 600 mL, about 650 mL, about 700 mL, about 750 mL, about 800 mL, about 850 mL, about 900 mL, about 950 mL, about 1000 mL, or any intermediate volumes therein. In certain embodiments, the ready-to-infuse oxytocin injection compositions are supplied in a fill volume of about 500 mL.

In certain embodiments, the ready-to-infuse oxytocin injection compositions of the disclosure are supplied in flexible plastic containers as opposed to rigid or semi-rigid containers. In certain embodiments, the flexible plastic container is a flexible bag or a flexible bottle.

In certain embodiments, the flexible plastic container is a closed system fully collapsible intravenous bag. The bag provides maximum drainage and minimum residual volume of the infusion liquid to minimize dosing error. Additionally, the bag does not require external venting to empty the bag.

In certain embodiments, the flexible plastic container is further packed in a secondary packaging material. In certain embodiments, secondary packaging material is an aluminum overwrap, aluminum over pouch or equivalent thereof. In certain embodiments, secondary packaging material is aluminum over pouch.

In certain embodiments, the flexible plastic container is a flexible bag comprising polyvinylchloride (PVC), polypropylene (PP), polyethylene (PE) or polyolefin blend (PP+PE).

In certain embodiments, the flexible plastic container is a flexible bag comprising a non-plasticized film containing polypropylene and thermoplastic elastomers (FREEFLEX®).

In certain embodiments, the flexible plastic container is a flexible bag comprising a multilayer polyolefin/styrene block copolymer-based film (POLY CINE®).

In certain embodiments, the flexible plastic container is a flexible bag comprising a multilayer sheeting composed of Polypropylene, Polyamide and Polyethylene (VIAFLO®).

In certain embodiments, the flexible plastic container is a flexible bag comprising a triple-layer double-wound PVC-free (Polypropylene based) film (MAGIFLEX®).

In certain embodiments, the flexible plastic container is a flexible bag comprising a multilayer film comprising polypropylene polymer or polypropylene based polyolefin polymer and styrene-ethylene-butylene block copolymer (TECHNOFLEX®).

In certain embodiments, the flexible plastic container is a flexible bag which is a non-diethylhexyl-phthalate (DEHP), non-polyvinyl chloride (PVC), and latex-free.

In certain embodiments, the ready-to-infuse oxytocin injection compositions of the disclosure are stabilized by minimizing the oxygen level in the composition. In certain embodiments, the oxygen level in the composition is minimized by manufacturing the solution composition in an inert atmosphere comprising an inert gas, e.g., nitrogen gas, to flush out any oxygen.

### Method of Manufacture

In another embodiment, the present disclosure provides methods for manufacturing the aqueous, ready-to-infuse oxytocin injection compositions. The method comprises: 1) preparing oxytocin solution in purified water, 2) Adding 900 ml of water for injection in a stainless steel container, adding pH adjusting agent/buffering agent, stabilizing agent, tonicity agent, and optionally, a preservative in the stainless steel container with continuous mixing, 3) adding the oxytocin solution from step 1 to the solution in step 2 with continuous mixing, 4) diluting the resulting solution with water for injection in QS to 1000 ml, and 5) storing the diluted solution from step 4 in a sealed flexible plastic container.

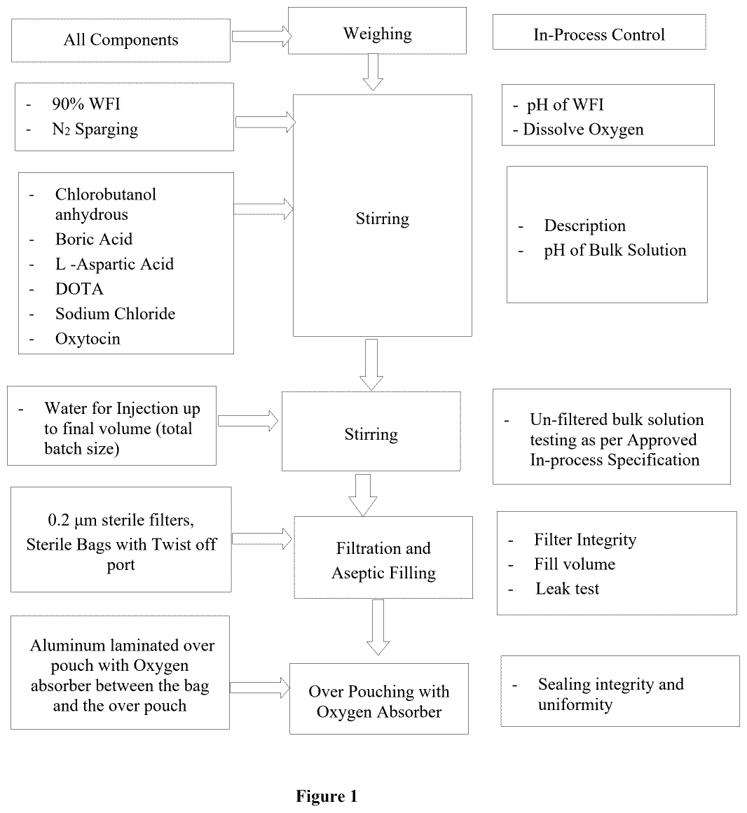

The ready-to-infuse compositions of the disclosure are made under continuous nitrogen purging and/or sparging. FIG. 1 provides a flow chart of the manufacturing process used for making ready-to-infuse oxytocin compositions of the disclosure.

In certain embodiments, the stainless-steel container is a closed container to avoid any evaporation of volatile excipients.

In certain embodiments, the flexible plastic container is a bag. In certain embodiments, the filled and sealed bags from step 5 are terminally sterilized and packed. In certain embodiments, the flexible plastic container is further packed in a secondary packaging material, e.g., aluminum overwrap, overpouch or equivalent thereof. In certain embodiments, the flexible plastic container is a flexible bag comprising a non-plasticized film containing polypropylene and thermoplastic elastomers (FREEFLEX®).

In certain embodiments, the flexible plastic container is a flexible bag comprising a multilayer polyolefin/styrene block copolymer-based film (POLYCINE®).

In certain embodiments, the flexible plastic container is a flexible bag comprising a multilayer sheeting composed of Polypropylene, Polyamide and Polyethylene (VIAFLO®).

In certain embodiments, the flexible plastic container is a flexible bag comprising a triple-layer double-wound PVC-free (Polypropylene based) film (MAGIFLEX®).

Other commercially available bags include bags sold under the trade names EXCEL®, VISIV®, NEXCEL®, INTERVIA®, SOLOMIX®, STEDIM 71®, STEDIM @100, VIAFLEX®, ADDEASE®, ADD-VANTAGE®, DUPLEX®, FIRST CHOICE®, PROPYFLEX®, and BFS®.

In certain embodiments, the flexible plastic container is a flexible bag comprising a multilayer film comprising polypropylene polymer or polypropylene based polyolefin polymer and styrene-ethylene-butylene block copolymer (TECHNOFLEX®).

In certain embodiments, the flexible plastic container is a flexible bag which is a non-DEHP (Di-ethylhexyl-phthalate), non-PVC (polyvinyl chloride) and latex-free.

In certain embodiments, the space between the flexible plastic container and the secondary packaging material is filled with inert gas. An inert gas may be used to flush or replace the air from the space between the flexible plastic container and the secondary packaging material. In certain embodiments, the empty headspace of the bag is flushed with inert gas to replace any oxygen in the head space of the bag. The inert gases that may be used include nitrogen, argon, and helium. In certain embodiments, the inert gases that may be nitrogen.

In certain embodiments, the dissolved oxygen content of the ready-to-infuse oxytocin solution composition of the disclosure is maintained at not more than (NMT) 2 ppm. In certain embodiments, the dissolved oxygen content of the ready-to-infuse oxytocin solution composition of the disclosure is maintained at less than 2 ppm, less than 1.5 ppm, less than 1 ppm, less the 0.5 ppm, or any intermediate values therein.

### Method of Treatment

Antepartum—The ready-to-infuse oxytocin solution compositions (oxytocin injection compositions) of the disclosure are used for initiation or improvement of uterine contractions, where this is considered desirable and suitable for reasons of fetal and maternal concerns and safety, in order to achieve vaginal delivery.

The ready-to-infuse oxytocin solution compositions of the disclosure are indicated for the following:
(1) Induction of labor in patients with a medical indication for the initiation of labor, such as Rh (Rhesus factor) problems, maternal diabetes, preeclampsia at or near term, when delivery is in the best interests of mother and fetus or when membranes are prematurely ruptured, and delivery is indicated.
(2) Stimulation or reinforcement of labor, as in selected cases of uterine inertia.
(3) As adjunctive therapy in the management of incomplete or inevitable abortion. In the first trimester, curettage is generally considered primary therapy. In second trimester abortion, oxytocin infusion will often be successful in emptying the uterus. Other means of therapy, however, may be required in such cases.

Postpartum—The ready-to-infuse oxytocin solution compositions of the disclosure are indicated to produce uterine contractions during the third stage of labor and control postpartum bleeding or hemorrhage.

Contraindications-Antepartum use of oxytocin solution compositions of the disclosure is contraindicated in any of the following circumstances:
1. Where there is significant cephalopelvic disproportion.
2. In unfavorable fetal positions or presentations, such as transverse lies, which are undeliverable without conversion prior to delivery.
3. In obstetrical emergencies where the benefit-to-risk ratio for either the fetus or the mother favors surgical intervention.
4. In fetal distress where delivery is not imminent.
5. Where adequate uterine activity fails to achieve satisfactory progress.
6. Where the uterus is already hyperactive or hypertonic.
7. In cases where vaginal delivery is contraindicated, such as invasive cervical carcinoma, active herpes genitalis, total placenta previa, vasa previa, and cord presentation or prolapse of the cord.
8. In patients with hypersensitivity to the drug.

### Dosing Regimen

The present disclosure provides injection compositions comprising from about 0.01 IU to about 0.1 IU oxytocin, as a premixed, ready-to-use solution for intravenousinfusion. No further dilution of these injection compositions is required. The ready-to-infuse oxytocin solution is inspected visually for particulate matter and discoloration prior to administration. The compositions can be administered at a rate of 1 mU/min to 10 mU/min, based on the dosage required for desired uterine response. In certain embodiments, the administration rate is about 1 mU/min, about 2 mU/min, about 3 mU/min, about 4 mU/min, about 5 mU/min, about 6 mU/min, about 7 mU/min, about 8 mU/min, about 9 mU/min, about 10 mU/min, or intermediate values therein. In certain embodiments, the administration rate is about 60 mU/hr, about 120 mU/hr, about 180 mU/hr, about 240 mU/hr, about 300 mU/hr, about 360 mU/hr, about 420 mU/hr, about 480 mU/hr, about 5400mU/hr, about 600 mU/hr, or intermediate values therein. In certain embodiments, the ready-to-use solution compositions of the disclosure contain about 10 mU/ml, about 15 mU/ml, about 20 mU/ml, about 25 mU/ml, about 30 mU/ml, about 35 mU/ml, about 40 mU/ml, about 45 mU/ml, about 50 mU/ml, about 55 mU/ml, about 60 mU/ml, about 65 mU/ml, about 70 mU/ml, about 75 mU/ml, about 80 mU/ml, about 85 mU/ml, about 90 mU/ml, about 95 mU/ml, about 100 mU/ml, or any intermediate values therein.

The dosage of oxytocin is determined by the uterine response and therefore is individualized and initiated at a very low level. The following dosage information is based upon various regimens and indications in general use.

Induction or Simulation of labor-Intravenous infusion (drip method) is the only acceptable method of parenteral administration of oxytocin injection for the induction or stimulation of labor.

Accurate control of the rate of infusion is essential and is best accomplished by an infusion pump.

It is convenient to piggyback the oxytocin injection infusion on a physiologic electrolyte solution, permitting the oxytocin injection infusion to be stopped abruptly without interrupting the electrolyte infusion. Attaching (Piggyback) of the oxytocin injection infusion on a physiologic electrolyte solution is done as follows:
1) Establishing infusion with a separate bottle of an electrolyte solution without oxytocin (placebo electrolyte solution)
2) Attach (piggyback) the oxytocin injection-containing bottle with the infusion pump to the infusion line as close to the infusion site as possible

Administration—An initial dose of administration should be 0.5-1 mU/min (equal to 3-6 mL/hr administration of injection solution containing 10 mU/mL oxytocin or 0.5-1 mL/hr administration of injection solution containing 60 mU/mL oxytocin). At 30-60 minute intervals the dose should be gradually increased in increments of 1-2 mU/min until the desired contraction pattern has been established. Once the desired frequency of contractions has been reached and labor has progressed to 5-6 cm dilation, the dose may be reduced by similar increments.

Oxytocin infusions have shown that infusion rates up to 6 mU/min give the same oxytocin levels that are found in spontaneous labor. At full pregnancy term, higher infusion rates should be given with great care, and rates exceeding 9-10 mU/min are rarely required. Before term, when the sensitivity of the uterus is lower because of a lower concentration of oxytocin receptors, a higher infusion rate may be required.

Monitoring—Uterine activity and the fetal heart rate are monitored throughout the infusion of oxytocin injection. Tonus, amplitude and frequency of contractions, and to the fetal heart rate in relation to uterine contractions are closely monitored. If uterine contractions become too powerful, the infusion should be stopped abruptly stopped to wane the oxytocic stimulation of the uterine musculature.

Postpartum uterine bleeding is minimized by adjusting the infusion rate of the oxytocin injection composition comprising 10-40 units of oxytocin in a bag (maximum 40 units in a 1000 mL bag) to sustain uterine contraction and control uterine atony.

Treatment of Incomplete, Inevitable, or Elective Abortion-Intravenous infusion of oxytocin injection (30 units of oxytocin in total of 500 mL) composition may help the uterus contract after a suction or sharp curettage for an incomplete, inevitable, or elective abortion. Subsequent to intra-amniotic injection of hypertonic saline, prostaglandins, urea, etc., for mid trimester elective abortion, the injection-to-abortion time may be shortened by infusion of oxytocin injection at the rate of 10 to 20 milliunits (6 to 13 drops) per minute. The total dose should not exceed 30 units in a 12-hour period due to the risk of water intoxication.

### Stability

Storage stability of the ready-to-infuse oxytocin solution compositions of the disclosure was compared among the compositions comprising stabilizers selected from amino acids, buffers, and chelating agents. The compositions were stored in glass vials and tested for stability up to 6 months (e.g., 1M, 2M, 3M, and 6M) under refrigerated conditions (e.g., from about 2 to about 8° C.); controlled room temperature conditions (CRT) (e.g., 25° C.±2° C. and 40±5% RH); and under accelerated storage conditions (Acc) (e.g., 40° C.±2° C. and 25±5% RH)

Compositions comprising one or more amino acids (e.g., L-aspartic acid) as a stabilizer; chelating agent (e.g., DOTA) and amino acid (e.g., L-aspartic acid) as a stabilizer; and chelating agent (e.g., DOTA) as a stabilizer were tested for stability and the results are summarized in the following Table 1.

TABLE 1

| | | 1 M | | | 3 M | | |
|---|---|---|---|---|---|---|---|
| Storage Cond's | Initial | Ref | CRT | Acc | Ref | CRT | Acc |
| L-Aspartic | | | | | | | |
| acid + DOTA | | | | | | | |
| % Assay | 104.7 | 97.8 | 96.7 | 92.2 | 95.6 | 91.9 | 81.2 |
| L-Aspartic | | | | | | | |
| % Assay | 93.3 | — | — | — | — | — | — |
| DOTA | | | | | | | |
| % Assay | 97.8 | — | — | — | — | — | — |

The data from Table 1 clearly demonstrates that initial assay % is maximum when the ready-to-infuse injection composition comprises DOTA and aspartic acid, compared to the compositions individually comprising aspartic or DOTA. The above data further demonstrates that the compositions comprising DOTA and aspartic acid contain at least about 90% oxytocin (90% assay) after storage under refrigerated storage conditions and storage at controlled room temperature conditions for at least about 3 months.

### EXAMPLES

Below are the examples of ready-to-infuse oxytocin injection compositions according to embodiments of the present disclosure.

### Example 1: Ready-to-Infuse Oxytocin Injection Composition

The following Table 2 provides oxytocin injection compositions 1 & 2.

TABLE 2

| | | | | | | | Maximum Quantity |
|---|---|---|---|---|---|---|---|
| Components | | Quantity | | Quantity/bag | | % Wt/Vol | Administered/MDD |
| Oxytocin | | 0.06 IU | | 30 | IU | 0.00001 | 60 IU (0.1 IU) |
| | | (0.0001 mg/mL) | | | | | |
| Chlorobutanol | | 0.25 | mg/mL | 1.25 mg/500 mL | | 0.025 | 2.5 |
| (anhydrous) | | | | | | | |
| Boric Acid | | 0.0121 | mg/mL | 0.05 mg/500 mL | | 0.00121 | 0.10 |
| L-Aspartic | Comp 1 | 0.02 | mg/mL | 10 | mg/ml | 0.002 | 20 |

| | | | | | | |
|---|---|---|---|---|---|---|
| Acid | Comp 2 | 0.047 | mg/mL | 23.5 mg/500 mL | 0.0047 | 47 |

| | | | | | |
|---|---|---|---|---|---|
| DOTA (Tetraxetan) | 0.005 | mg/mL | 2.5 mg/500 mL | 0.0005 | 5 |
| Sodium Chloride | 9 | mg/mL | 4500 mg/500 mL | 0.9 | 9000 |

| | | | | |
|---|---|---|---|---|
| Water for | Q.S. to 1 mL | Q.S. to 500 mL | Q.S. | Q.S. to |
| Injection (WFI) | | | | 1000 mL |

### Manufacturing Process

The following method for manufacturing and packaging of the ready-to-infuse oxytocin solution was conducted under continuous nitrogen purging:
1) A bout 900 ml of water for injection was added to a stainless-steel container/manufacturing vessel.
2) Chlorobutanol was added to the vessel from step 1 and stirred with heating at a temp of about 45° C.-55° C. until a clear solution was obtained. The resulting clear solution was cooled to room temperature (about 25° C.±2° C.).
3) Boric acid was added to the cooled solution from step 2 and stirred until a clear solution was obtained.
4) Aspartic acid was added to the solution from step 3 and stirred until a clear solution was obtained.
5) DOTA was added to the solution from step 4 and stirred until a clear solution was obtained.
6) 110 mg of oxytocin was added to the solution from step 5 and stirred to obtain a clear solution.
7) Sodium chloride was added to the solution from step 6 and stirred to obtain a clear solution.
8) The resulting solution from step 7 was diluted with water for injection in QS to 1000 ml to provide 0.06 IU/ml oxytocin solution.
9) The diluted solution from step 8 was filtered using 0.2 μ sterile filter and filled in a flexible plastic container.

### Example 2: Effect of Aspartic Acid Amount on Storage Stability of Ready-to-Infuse Oxytocin Injection Compositions

Initial % assay, and one month and three-month storage stability of the ready-to-infuse oxytocin solution compositions from Example 1 (Compositions 1 and 2 containing 0.047 mg/ml aspartic acid and 0.02 mg/ml aspartic acid, respectively) was determined under different storage conditions (e.g., controlled room temp, refrigerated conditions, and accelerated storage conditions). The compositions were stored in glass vials and tested for 3M storage stability under refrigerated conditions (e.g., from about 2° C.-8° C.); controlled room temperature conditions (CRT) (e.g., 25° C.±2° C. and 40±5% RH); and accelerated storage conditions (Acc) (e.g., 40° C.±2° C. and 25±5% RH)

Table 3 provides Initial. 1M, and 3M storage stability of oxytocin compositions 1 and 2 under refrigerated conditions; controlled room temperature conditions; and accelerated storage conditions.

TABLE 3

| | | 1 M | | | 3 M | | |
|---|---|---|---|---|---|---|---|
| Storage Cond's | Initial | Ref | CRT | Acc | Ref | CRT | Acc |
| % Assay of | 100.7 | 94.3 | 92.0 | 96.1 | 93.8 | 82.8 | 93.9 |
| Composition 1 | | | | | | | |
| % Assay of | 106.4 | 101.7 | 98.2 | 102.4 | 100.8 | 91.9 | 103.1 |
| Composition 2 | | | | | | | |

The data from Table 3 shows that presence of 0.02 mg/ml of aspartic acid (Composition 1) is sufficient to provide at least about 90% assay after storage under refrigerated, controlled room temperature, and accelerated storage conditions up to about 3 months.

### EXAMPLE 3: Stability of Ready-to-Infuse Oxytocin Injection Compositions

The following Table 4 provides oxytocin injection compositions 3-5.

TABLE 4

| | | Chloro- | Boric | Aspartic | | Sod | | Initial |
|---|---|---|---|---|---|---|---|---|
| Comp # | Oxytocin | butanol | Acid | Acid | DOTA | Chloride | WFI | % assay |
| 3. | 0.06 | 0.025% | 0.00121 | 0.0047 | 0.0005 | 0.9% | q.s. | 100.1 |
| | IU/mL | w/v | w/v | w/v | w/v | w/v | | |
| 4. | 0.06 | 0.025% | 0.00121 | 0.0047 | NA | 0.9% | q.s. | 93.3 |
| | IU/mL | w/v | w/v | w/v | | w/v | | |
| 5. | 0.06 | 0.025% | 0.00121 | NA | 0.0005 | 0.9% | q.s. | 97.8 |
| | IU/mL | w/v | w/v | | w/v | w/v | | |

Composition 3-5 were made according to the general procedure described in Example 1/FIG. 1. Composition 3 contained DOTA and aspartic acid; Composition 4 contained aspartic acid and no DOTA; and Composition 5 contained DOTA and no aspartic acid. The data from Table 4 demonstrates that compositions comprising oxytocin, DOTA, and aspartic acid (Comp 3) provided maximum initial % assay, followed by the compositions with DOTA and without aspartic acid (Comp 5), and finally the compositions with aspartic acid and without DOTA (Comp 4) had the lowest initial % assay of oxytocin. The data clearly demonstrates that the presence of DOTA improves initial assay of oxytocin.

### Example 4: Storage Stability of Oxytocin Injection Compositions Without Aspartic Acid

TABLE 5

| | 1 Month | | | 2 Month | | | 3 Month | | |
|---|---|---|---|---|---|---|---|---|---|
| Initial | CRT | ACC | REF | CRT | ACC | REF | CRT | ACC | REF |
| 104.2 | 91.9 | 78.8 | 102.1 | 90.7 | 78.4 | 102.1 | 85.4 | 69.4 | 102.2 |

Table 5 provides storage stability of oxytocin composition 5 (Comp 3 without aspartic acid) under controlled room temperature (CRT), accelerated storage conditions (ACC), and refrigerated storage conditions (REF). The above data clearly shows aspartic acid improves storage stability of oxytocin. The assay of oxytocin drops in the absence of aspartic acid under all three storage conditions.

### Example 5: Storage Stability of a Ready-to-Dilute Product Diluted to Provide Oxytocin Concentration of 0.06 IU/mL

TABLE 6
Ready to Dilute Oxytocin Product (FDA
Approved and Marketed Product)

| Components | Quantity/mL | |
|---|---|---|
| Oxytocin | 10 | IU/mL |
| Chlorobutanol (anhydrous) | 5 | mg/mL |

| | |
|---|---|
| Acetic acid | 1.65 mg (QS to pH 3.5) |
| Ammonium acetate | 0.16 |
| Water for Injection | Q.S. |

Table 6 provides a composition of the FDA (Food and Drug Administration) approved ready-to-dilute marketed oxytocin product. The marketed product was diluted to a concentration of about 0.06 IU/mL. The diluted product was then tested for storage stability under refrigerated storage conditions (from about 2° C. to about 8° C.).

Table 7 provides % assay of oxytocin after storage under refrigerated storage conditions for a 14 days.

TABLE 7

| Days | % Assay of Oxytocin |
|---|---|
| Initial | 97.2 |
| 2 | 90.1 |
| 6 | 88 |
| 14 | 39.9 |

Data from Table 7 shows that the marketed product after dilution to a concentration of about 0.06 IU/mL showed a substantial decrease in % assay of oxytocin at day 14.

### Example 6: Effect of Bag on Storage Stability of Oxytocin Injection Composition 1 (Example 1)

Table 8 provides one month storage stability of Oxytocin Composition 1 (Example 1) in polyethylene bag and polypropylene bag, when stored under controlled room temperature and accelerated storage conditions.

TABLE 8

| | % Oxytocin Assay | | |
|---|---|---|---|
| | | CRT | ACC |
| Polymer | Initial | (25° C. ± 5° C.) | (40° C. ± 5° C.) |
| Polyethylene (PE) | 101 | 96.2 | 93.5 |
| Polypropylene (PP) | 101 | 92.1 | 88.6 |

Data from Table 8 clearly demonstrates that ready-to-infuse oxytocin compositions of the disclosure are stable when stored in a polyethylene bag or a polypropylene bag.

### Example 7: Storage Stability of Additional Oxytocin Injection Compositions

The following Table 9 provides oxytocin injection compositions 6-8.

TABLE 9

| | Oxytocin | Boric Acid | Aspartic | DOTA | Sod Chloride | |
|---|---|---|---|---|---|---|
| Comp | IU/ml | mg/ml | Acid mg/ml | mg/ml | mg/ml | |
| # | (% wt/vol) | (% wt/vol) | (% wt/vol) | (% wt/vol) | (% wt/vol) | WFI |
| 6. | 0.01 | 0.0121 | 0.005 | 0.002 | 9 | q.s. |
| | (1 Unit) | (0.00121) | (0.0005) | (0.0002) | (0.9) | |
| 7. | 0.02 | 0.0121 | 0.01 | 0.004 | 9 | q.s. |
| | (2 Units) | (0.00121) | (0.001) | (0.0004) | (0.9) | |
| 8. | 0.04 | 0.0121 | 0.02 | 0.004 | 9 | q.s. |
| | (4 Units) | (0.00121) | (0.002) | (0.0004) | (0.9) | |

Compositions 6-8 were made according to the general procedure described in Example 1/FIG. 1. Table 10 provides assay, density, viscosity, pH, and osmolality before (initial) and after storage of compositions 6-8 under controlled room temperature and accelerated storage conditions.

TABLE 10

| Composition | Storage | | | | | |
|---|---|---|---|---|---|---|
| Number | Condition | Assay | Density | Viscosity | pH | Osmolality |
| 6 | Initial | 98.3 | 1.004 | 0.93 | 4.63 | 288 |
| | CRT/1 M | 93.6 | 1.004 | 0.94 | 4.65 | 285 |
| | ACC/1 M | 88.3 | 1.003 | 0.94 | 4.54 | 291 |
| 7 | Initial | 95.7 | 1.004 | 0.94 | 4.61 | 288 |
| | CRT/1 M | 95.3 | 1.004 | 0.92 | 4.47 | 287 |
| | ACC/1 M | 93.7 | 1.004 | 0.93 | 4.43 | 285 |
| 8 | Initial | — | 1.004 | 0.93 | 4.45 | 287 |
| | CRT/1 M | 96.7 | 1.004 | 0.92 | 4.47 | 287 |
| | ACC/1 M | 96.9 | 1.004 | 0.94 | 4.07 | 288 |

TABLE 11

| | Composition 6 | | | Composition 7 | | | Composition 8 | | |
|---|---|---|---|---|---|---|---|---|---|
| | Storage Condition | | | Storage Condition | | | Storage Condition | | |
| | | CRT | ACC | | CRT | ACC | | CRT | ACC |
| Impurity Type | Initial | 1 M | 1 M | Initial | 1 M | 1 M | Initial | 1 M | 1 M |
| Leu3 Oxytocin | ND | ND | ND | ND | ND | ND | 0.00 | ND | ND |
| (NMT 0.3%) | | | | | | | | | |
| Oxytocic acid | ND | ND | ND | ND | 0.07 | 0.43 | 0.00 | 0.23 | 0.96 |
| (NMT 4.0%) | | | | | | | | | |
| Asp5_Oxytcoin | ND | ND | ND | ND | ND | ND | 0.00 | ND | ND |
| (NMT 0.3%) | | | | | | | | | |
| Glu Oxytocin | ND | ND | ND | ND | ND | 0.42 | 0.00 | 0.24 | 0.91 |
| (NMT5.0%) | | | | | | | | | |
| BOC Oxytocin | ND | ND | ND | ND | ND | ND | 0.00 | ND | ND |
| (NMT 0.3%) | | | | | | | | | |
| AC Oxytocin | 0.64 | 0.69 | 0.76 | 0.65 | 0.82 | 0.74 | 0.76 | 0.78 | 0.74 |
| (NMT 5.0%) | | | | | | | | | |
| Dimer | ND | ND | ND | ND | ND | ND | 0.00 | ND | ND |
| (NMT 1.0%) | | | | | | | | | |
| % Known | 0.64 | 0.69 | 0.76 | 0.65 | 0.89 | 1.59 | 0.76 | 1.25 | 2.61 |
| Impurity | | | | | | | | | |
| Unknown | ND | ND | ND | ND | ND | ND | ND | ND | ND |
| Impurity | | | | | | | | | |
| (NMT 0.3%) | | | | | | | | | |
| Total Impurity | 0.64 | 0.69 | 0.76 | 0.65 | 0.89 | 1.59 | 0.76 | 1.25 | 2.61 |
| (NMT 16.0%) | | | | | | | | | |

Data from Tables 10 and 11 clearly demonstrate that ready-to-infuse oxytocin compositions 6-8 are stable under controlled room temperature and accelerated storage conditions for up to about 1 month.

1. An aqueous, ready-to-infuse oxytocin injection composition comprising from about 0.01 IU/mL to 0.1 IU/ml of oxytocin, a chelating agent, and an amino acid,
wherein the composition comprises oxytocin: chelating agent wt/vol ratio of from about 1:10 to about 1:750,
wherein the composition contains at least about 90% wt/vol of oxytocin assay after storage for at least about 3 months under refrigerated and controlled room temperature conditions.

2. The composition of claim 1, wherein the composition comprises a wt/vol ratio of oxytocin:
amino acid of from about 1:50 to about 1:2000.

3. The composition of claim 1, wherein the refrigerated conditions comprise a temperature of about 2-8° C. and controlled room temperature conditions comprise a temperature of 25° C.±5° C. and 40%±5% RH.

4. The composition of claim 1, wherein the chelating agent is selected from the group consisting of 1,4,7,10-tetraazacyclododecane-1,4,7,10-tetraacetic acid, diethylene triamine-N,N,N′,N″,N″-pentaacetate/pentetic acid, ethylenediamine tetraacetic acid, calcium disodium edetate or their salts, and mixtures thereof.

5. The composition of claim 4, wherein the chelating agent is 1,4,7,10-tetraazacyclododecane-1,4,7,10-tetraacetic acid.

6. The composition of claim 1, wherein the amino acid is selected from the group consisting of arginine, glycine, alanine, proline, methionine, lysine, leucine, cysteine, aspartic acid, isoleucine, and mixtures thereof.

7. The composition of claim 1, wherein amino acid is L-aspartic acid.

8. The composition of claim 1, wherein the composition further comprises sodium chloride.

9. The composition of claim 1, wherein the composition further comprises a buffer selected from the group consisting of phosphate buffer, citrate buffer, sodium carbonate, sodium bicarbonate, tartarate, benzoate, ascorbic acid, succinic acid, lactic acid, glutaric acid, malic acid, boric acid, orthophosphoric acid and carbonic acid, alkali or alkaline earth salt of one of these acids, and mixtures thereof.

10. The composition of claim 1, wherein the composition does not include a preservative.

11. The composition of claim 1, wherein the composition exhibits a pH of between 3.5 and 5.5.

12. An aqueous, ready-to-infuse oxytocin injection composition comprising from about 0.01 IU to 0.1 IU/ml of oxytocin, a chelating agent, and aspartic acid,
wherein the composition comprises oxytocin: aspartic acid wt/vol. ratio of from about 1:50 to about 1:2000,
wherein the composition does not include a preservative, and
wherein the composition contains at least about 90% wt/vol of oxytocin assay after storage for at least about 3 months under refrigerated and controlled room temperature conditions.

13. The composition of claim 12, wherein the oxytocin: chelating agent ratio is from about 1:10 to about 1:750.

14. The composition of claim 12, wherein the composition contains at least about 95% wt/vol of oxytocin assay after storage for at least about 3 months under refrigerated and controlled room temperature conditions.

15. The composition of claim 12, wherein the refrigerated conditions comprise about 2-8° C. and the controlled room temperature conditions comprise 25° C.±2° C. and 40%±5% RH.

16. The composition of claim 12, wherein the composition comprises from about 0.000016 mg/ml to about 0.0001 mg/ml of oxytocin.

17. The composition of claim 12, wherein the composition further comprises a buffer selected from the group consisting of phosphate buffer, citrate buffer, sodium carbonate, sodium bicarbonate, tartrate, benzoate, ascorbic acid, succinic acid, lactic acid, glutaric acid, malic acid, boric acid, orthophosphoric acid and carbonic acid, alkali or alkaline earth salt of one of these acids, and mixtures thereof.

18. The composition of claim 12, wherein the chelating agent is 1,4,7,10-tetraazacyclododecane-1,4,7,10-tetraacetic acid.

19. The composition of claim 12, wherein the composition exhibits a pH of between 3.5 and 5.5.

20. A n aqueous, ready-to-infuse oxytocin injection composition comprising from about 0.01 IU to 0.1 IU/mL of oxytocin, a chelating agent, and an amino acid, wherein
the composition comprises oxytocin: aspartic acid wt/vol ratio of from about 1:50 to about 1:2000,
the composition comprises oxytocin: chelating agent wt/vol ratio of from about 1:10 to about 1:750,
wherein the composition contains at least about 90% wt/vol of oxytocin assay after storage for at least about 3 months under refrigerated and controlled room temperature conditions.

21. The composition of claim 19, wherein the composition contains at least about 95% wt/vol of oxytocin assay after storage for at least about 3 months under refrigerated and controlled room temperature conditions.
